(19) United States
(12) Patent Application Publication
(10) Pub. No.: US 2005/0090687 A1
(43) Pub. Date: Apr. 28, 2005

(54) Method for producing carboxylic acids by alcohol oxidation

(76) Inventors: Paul Alsters, Maastricht (NL); Sabine Bouttemy, Toutencourt (FR); Elisabeth Schmieder-Van De Vondervoort, Haelen (NL); Jose Manuel Padron Carillo, Geleen (NL)

Correspondence Address: WENDEROTH, LIND & PONACK, L.L.P., 2033 K STREET N. W., SUITE 800, WASHINGTON, DC, 20006-1021, US

(21) Appl. No.: 10/990,521

(22) Filed: Nov. 18, 2004

Related U.S. Application Data
(63) Continuation of application No. 10/168,849, filed on Jun. 26, 2002, now abandoned.

(30) Foreign Application Priority Data
Jan. 19, 2000 (AT) A 79/2000

(51) Int. Cl. C07C 227/02; C07C 225/06

(52) U.S. Cl. 562/538; 564/502

(57) ABSTRACT

A method for oxidizing primary amino alcohol, primary or secondary alkenol or alkynol to the corresponding acid or ketone, wherein a primary amino alcohol, a primary or secondary alkenol or alkynol as a substrate is oxidized to the corresponding ketone in the presence of an equimolar amount or a molar excess, based on the alcoholic hydroxyl groups, of periodate, catalytic amounts of dichromate or CrO₃, and in the presence of an acid, in water, a water/solvent mixture or in a solvent at a temperature of −20° C. to +50° C. to give the corresponding acid or corresponding ketone.

The invention relates to a method for oxidizing amino alcohols, primary or secondary alkenols or alkynols to the corresponding carboxylic acids or ketones.

Oxidation is a fundamental transformation in organic synthesis, so that numerous methods have already been described for it in the literature. Nevertheless, direct conversion of primary alcohols to the corresponding carboxylic acids, in particular in the presence of other functional groups or double or triple bonds, is still associated with problems. For these reactions there are to date no, or only a few, useful methods, which use, for example, CrO₃/H₂SO₄, RuCl₅/H₅IO₆ or TEMPO/NaClO as reagents. However, these variants all have limitations and disadvantages, so that novel oxidation methods are still being sought. Tetrahedron Letters 39 (1998) 5323-5326 describes, for example, the oxidation of primary alcohols to carboxylic acids using periodic acid H₅IO₆ as a stoichiometric oxidant and catalytic amounts of CrO₃. Reference is made here to the fact that the best results are achieved when MeCN containing traces of water is used as solvent and the reaction temperature is 0 to 5° C. Further, it was found that no reaction was observed when the periodic acid was replaced by other oxidizing agents. However, the disadvantage with this method is that when, for example, amino alcohols are used as starting material, the amino group must be protected by a suitable protecting group such as benzyloxycarbonyl (Cbz). This requires an additional outlay, since the amino group must be protected against oxidation using a protecting group which must be removed again after the reaction is complete.

It was an object of the invention to find a suitable method for oxidizing amino alcohols and of primary and secondary alkenols or alkynols to the corresponding carboxylic acids or ketones, in which method the amino group need not be protected by introducing an amino protecting group and which ensures a high conversion rate of the alkenols and alkynols.

Unexpectedly, this object has been achieved by using periodate in combination with dichromate or CrO₃ in the presence of an acid.

The invention therefore relates to a method for oxidizing primary amino alcohols, primary or secondary alkenols or alkynols to the corresponding acids or ketones which is characterized in that a primary amino alcohol, a primary or secondary alkenol or alkynol as substrate is oxidized to the corresponding ketone in the presence of an equimolar amount or a molar excess, based on the alcoholic hydroxyl groups, of periodate, catalytic amounts of dichromate or CrO₃, and in the presence of an acid, in water, a water/solvent mixture or in a solvent at a temperature of −20° C. to +50° C. to give the corresponding acid or corresponding ketone.

In the inventive method, primary amino alcohols, primary or secondary alkenols or alkynols are oxidized to the corresponding acids or ketones.

Amino alcohols are taken to mean compounds which not only have amino groups but also alcoholic hydroxyl groups as functional groups.

Primary and secondary alkenols and alkynols are given to mean compounds which have one or two primary or secondary alcoholic hydroxyl groups as functional groups and one or more double or triple bonds.

Suitable amino alcohols, alkenols or alkynols are compounds of the formula I 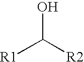
where R1 is either H or a C₁-C₂₀ alkyl radical, an aryl or heteroaryl radical or a heterocycle and R2 is an unbranched or branched, unsubstituted or substituted C₂-C₂₀ alkenyl or alkynyl radical or a C₁-C₂₀ alkyl or aryl radical substituted by one or two amino groups.

Alkyl radicals are taken to mean unbranched, branched or cyclic alkyl groups. These radicals can be unsubstituted or substituted by one or more substituents inert under the reaction conditions, such as acyl, carboxyl, halogen, C₁-C₈ alkoxy, C₃-C₈ cycloalkyl, phenyl, naphthyl, heteroaryl, heterocycle, etc.

Aryl is taken to mean phenyl or naphthyl which in turn are unsubstituted or are substituted by acyl, carboxyl, halogen, C₁-C₈ alkoxy, C₃-C₈ cycloalkyl, etc.

Heteroaryl radicals are 5- or 6-membered aromatic rings which have 1 to 3 heteroatoms selected from the group consisting of O, N or S. These radicals can also be unsubstituted or substituted by acyl, carboxyl, halogen, C₁-C₈ alkoxy, C₃-C₈ cycloalkyl, etc. In addition, the heteroaryl radicals can be present as benzocondensed ring systems, which can also be substituted as described above.

Heterocyclic radicals are 5- or 6-membered non-aromatic rings which have 1 to 3 heteroatoms selected from the group consisting of O, N or S. These radicals can in turn be unsubstituted or substituted by acyl, carboxyl, halogen, C₁-C₈ alkoxy, C₃-C₈ cycloalkyl, etc. In addition, the heterocyclic radicals can also be present as benzocondensed ring systems, which can also be substituted as described above.

Preferred amino alcohols are aliphatic or aromatic amino alcohols having 2 to 20 carbon atoms which have 1 to 2 amino groups and 1 to 2 primary hydroxyl groups, so that R1 is H. If appropriate the compounds can be substituted by further substituents inert under the reaction conditions, for instance acyl, carboxyl, halogen, C₁-C₈ alkoxy, phenyl, etc. The amino alcohols can also be monosubstituted or disubstituted on the amino group, for example by C₁-C₈ alkyl groups or unsubstituted or substituted aryl groups.

The preferred aliphatic amino alcohols can have not only an unbranched, but also a branched alkyl moiety which can be unsubstituted or substituted by acyl, carboxyl, halogen, C₁-C₈ alkoxy, phenyl, etc. Examples of these are 2-amino-1-ethanol, 2-amino-2-phenyl-ethanol, 2-aminopropanol, 2-aminohexanol, 3-amino-1-propanol, 2-amino-2-methyl-1-propanol, 2-amino-2-methyl-1,3-propanediol, 2-amino-3-phenyl-1-propanol, 2-amino-1-butanol or N-substituted amino alcohols, for instance N-methyl, N,N-diethyl-N,N-diisopropyl or N,N-dibutylaminoethanol, N-acetyl-2-amino-3-phenyl-propanol (acetylphenylalaninol) or N-phenylamino-ethanol.

Preferred primary and secondary alkenols and alkynols are compounds of the formula I where R1 is H or an unbranched or branched C₁-C₈ alkyl radical and R2 is C₃-C₁₂ alkenyl or alkynyl radical having one or more double or triple bonds. The radicals are preferably unbranched or branched and can be unsubstituted or substituted by one or more substituents which are inert under the reaction conditions, such as acyl, carboxyl, halogen, C₁-C₈ alkoxy, C₃-C₈ cycloalkyl, phenyl, etc. Preferably, the alkenyl and alkynyl radicals are unsubstituted. Examples of these are 3-heptyn-1-ol, 4-heptyn-2-ol, 3-hexyn-2-ol, 3-pentyn-1-ol, 3-butyn-1-ol, 4-methyl-3-penten-1-ol, 3-buten-1-ol, trans-3-hexen-1-ol, 5-hexyn-3-ol, 3-phenyl-2-propen-1-ol.

The inventive oxidation of the alcohols is performed in the presence of an equimolar amount, or a molar excess, based on the alcoholic hydroxyl groups present in the substrate, of periodate. Preferably, 1.5 to 10 molar equivalents, particularly preferably 2 to 5 molar equivalents, of periodate are used. Periodate is used as Na, K or Bu₄N salt, sodium periodate being preferred.

In addition, for the inventive oxidation, dichromate or CrO₃ is added in catalytic amounts. Suitable dichromates are Na dichromate or K dichromate. Preferably, sodium dichromate is used. The amount of dichromate or CrO₃ is about 0.1 to 3 mol %, based on the substrate. Preferably an amount of 0.3 to 2 mol % of dichromate or CrO₃ is added.

As third component an acid is added. Suitable acids here are sulfuric acid, HCl, HNO₃, p-toluenesulfonic acid (p-TSA), HBF₄, H₅IO₆, CF₃SO₃H or perfluorotetradecanoic acid (PFTDA) or mixtures thereof. Preferred acids are H₂SO₄, HNO₃ and H₅IO₆ and mixtures thereof.

The acid is used in the oxidation of amino alcohols in an equimolar amount or in a molar excess, based on the amino groups. Preferably, in the oxidation of the amino alcohols, an amount of acid of 1 to 4 molar equivalents, particularly preferably 1.1 to 2 molar equivalents, is used. In the case of alkenols and alkynols, preferably an amount corresponding to 1-30 mol % of H⁺, preferably 5-20 mol % of H⁺, of acid is used.

The inventive oxidation is performed in water, in a solvent or in a water/solvent mixture. Suitable solvents are chloroform, dichloromethane, ethyl acetate, diethyl ether, methyl t-butyl ether, dimethoxyethane, 2-methoxyethyl ether, triethylene glycol dimethyl ether, dioxane, THF, acetone, isopropyl acetate and acetonitrile.

In the oxidation of the amino alcohols the three oxidation components periodate, dichromate or CrO₃, and acid are preferably dissolved in water. The substrate to be oxidized is then added with stirring. The substrate can be added as such or if appropriate as solution in one of the above-described solvents or water/solvent mixture.

In the case of the alkenols and alkynols, dichromate, or CrO₃, and periodate are introduced and stirred in the water bath. Water, an above-described solvent or a water/solvent mixture and the corresponding starting material and the acid are then added.

The reaction temperature in both variants, depending on the solvent system selected, is −20° C. to +50° C., preferably −10 to +30° C., and particularly preferably 0 to 25° C.

If a two-phase system is employed, the reaction mixture is stirred vigorously during the entire reaction. If only an aqueous phase is employed, the vigorous stirring may not be necessary.

The reaction time depends on the substrate used and is between 1 and 40 hours. Preferably, the reaction time is between 6 and 30 hours, particularly preferably between 12 and 25 hours.

If appropriate, after part of the reaction time, a further portion of periodate and/or acid can be added to the reaction mixture in order to complete the oxidation to the carboxylic acid or ketone.

At the end of the oxidation, the corresponding carboxylic acid or ketone is isolated from the reaction mixture. Depending on the physical state, this is performed by conventional methods, for example by extraction, filtration, etc.

The remaining reaction solution can be worked up to regenerate the periodate. This can be performed by methods known from the literature, for example by chemical or electrochemical oxidation. Preferably, the periodate is regenerated by ozone, as described, for example in WO 98/27118. The regenerated periodate can then be reused for further oxidations.

By means of the inventive method, the amino alcohols and the primary and secondary alkenols and alkynols can be converted to the corresponding carboxylic acids or ketones, depending on the reaction time, up to a rate of 95% and above. Unreacted alcohols may readily be separated off from the end product during its isolation.

A further advantage of the method is the simple reaction procedure, with it being in particular advantageous that the amino group of the substrate used need not be protected by a protecting group, which thus does not need to be removed after the reaction is completed.

### EXAMPLE 1

### 4-Aminobutanoic Acid

0.47 g of sodium periodate NaIO₄ (2.2·10⁻³ mol), 1.6 mg of sodium dichromate Na₂Cr₂O₇ (5.4·10⁻⁶ mol) and 0.11 g of sulfuric acid H₂SO₄ (1.1·10⁻³ mol) were dissolved in 3 ml of water. To this solution were added 94.5 mg of 4-amino-1-butanol (1.06·10⁻³ mol) whereupon the reaction mixture was stirred vigorously for 17 h at 20° C. After 17 h the reaction solution was analyzed by ¹H NMR. Comparison with the NMR spectrum of commercially available 4-aminobutanoic acid showed a conversion rate to 4-aminobutanoic acid of 94%. The ratio of alcohol to carboxylic acid was therefore 6:94.

### EXAMPLE 2

### Phenylalanine

0.49 g of sodium periodate NaIO₄ (2.3·10⁻³ mol), 2.4 mg of sodium dichromate Na₂Cr₂O₇ (8.3·10⁻⁶ mol) and 0.12 g of sulfuric acid H₂SO₄ (1.2·10⁻³ mol) were dissolved in 2 ml of water. To this solution was added 0.14 g of phenylalaninol (0.9·10⁻³ mol) dissolved in chloroform, whereupon the two-phase system was stirred vigorously for 20 h at 20° C. After 20 h both phases were analyzed by ¹H NMR. Comparison with the NMR spectrum of commercially available phenylalanine showed, for the aqueous phase, a conversion rate to 4-phenylalanine of 44%. The ratio of alcohol to carboxylic acid in the aqueous phase was therefore 56:44.

Water and chloroform were added to work up the reaction mixture. The organic phase was extracted once with water. The combined aqueous phases were concentrated on a rotary evaporator, whereupon 0.586 g of a yellowish-green substance were obtained which comprised the product, unreacted alcohol, sodium periodate and Cr catalyst. NMR analysis of the mixture found 62% phenylalanine and 38% phenylalaninol.

### EXAMPLE 3

### Phenylalanine

0.29 g of sodium periodate NaIO₄ (1.34·10⁻³ mol), 2.9 mg of sodium dichromate Na₂Cr₂O₇ (1.0·10⁻⁵ mol) and 73 mg of sulfuric acid H₂SO₄ (7.5·10⁻⁴ mol) were dissolved in 3 ml of water. To this solution were added 75.6 mg of phenylalaninol (0.5·10⁻³ mol), whereupon the reaction mixture was stirred vigorously for 20 h at 20° C. After 20 h the reaction solution was analyzed by ¹H NMR. The ratio of phenylalaninol to phenylalanine was 25:75.

### EXAMPLE 4

### 2-Amino-1-propanoic Acid

0.47 g of sodium periodate NaIO₄ (2.2·10⁻³ mol), 3.5 mg of sodium dichromate Na₂Cr₂O₇ (1.17·10⁻⁵ mol) and 0.13 g of sulfuric acid H₂SO₄ (1.3·10⁻³ mol) were dissolved in 3 ml of water. To this solution were added 72.8 mg of 2-amino-1-propanol (0.97·10⁻³ mol), whereupon the reaction mixture was stirred vigorously for 20 h at 20° C. After 20 h the reaction solution was analyzed by ¹H NMR. The ratio of 2-amino-1-propanol to 2-amino-1-propanoic acid was 63:37. A further 0.47 g of sodium periodate NaIO₄ (2.2·10⁻³ mol) was then added and the mixture was stirred for a further 4 h. Renewed NMR analysis gave a conversion rate of 72%.

### EXAMPLE 5

### 3-Amino-1-propanoic Acid

0.51 g of sodium periodate NaIO₄ (2.4·10⁻³ mol), 2.4 mg of sodium dichromate Na₂Cr₂O₇ (8.0·10⁻⁶ mol) and 0.17 g of sulfuric acid H₂SO₄ (1.7·10⁻³ mol) were dissolved in 3 ml of water. To this solution were added 75 mg of 3-amino-1-propanol (1.0·10⁻³ mol), whereupon the reaction mixture was stirred vigorously for 20 h at 20° C. After 20 h, the reaction solution was analyzed by ¹H NMR. The ratio of 3-amino-1-propanol to 3-amino-1-propanoic acid was 5:95. NMR analysis after 4 h of reaction time had already found a conversion rate of 85%.

### EXAMPLE 6

### N-Acetylphenylalanine

0.47 g of sodium periodate NaIO₄ (2.2·10⁻³ mol), 2.1 mg of sodium dichromate Na₂Cr₂O₇ (7.0·10⁻⁶ mol) and 0.12 g of sulfuric acid H₂SO₄ (1.2·10⁻³ mol) were dissolved in 3 ml of water. To this solution was added 0.15 g of N-acetylphenylalaninol (0.78·10⁻³ mol), whereupon the reaction mixture was stirred vigorously for 20 h at 20° C. After 20 h the reaction solution was analyzed by ¹H NMR. Analysis found complete conversion, and comparison with commercially available N-acetyl-phenylalanine confirmed the formation of N-acetyl-phenylalanine.

### EXAMPLE 7

1 mol % of sodium dichromate (2 mol % of Cr) or 2 mol % of CrO₃, and 2.2 equivalents of sodium periodate were introduced into a reaction vessel which was situated in a 20° C. water bath. The mixture was stirred using a magnetic stirrer. Water (D₂O), solvent and acid (equivalent to 20 mol % H⁺) and also 1 mmol (112 mg) of 3-heptyn-1-ol were then added and the reaction mixture was stirred at a temperature between 0° C. and 30° C. After the time given in the table the reaction mixture was filtered to remove insoluble sodium iodate and isolate 3-heptynoic acid.

The amounts used and the reaction parameters (temperature, reaction time and yield) are cited in table 1:

TABLE 1

| ml | ml | | | 2 mol % | T | | % |
|---|---|---|---|---|---|---|---|
| (solvent) | D₂O | Periodate | Acid | of Cr | (° C.) | t (h) | yield |
| 2 (CD₃CN) | 1 | 2.2 eq. NaIO₄ | H₂SO₄ | Na₂Cr₂O₇ | 20 | 18 | 81 |
| 2 (CD₃CN) | 1 | 2.2 eq. NaIO₄ | H₂SO₄ | Na₂Cr₂O₇ | 20 | 16 | 83 a) |
| 2 (CD₃CN) | 1 | 2.2 eq. NaIO₄ | H₅IO₆ | Na₂Cr₂O₇ | 20 | 3 h 30′ | 73 |
| 2 (CD₃CN) | 1 | 2.2 eq. NaIO₄ | HNO₃ | Na₂Cr₂O₇ | 20 | 7 | 80 |
| 2 (CD₃CN) | 1 | 2.2 eq. NaIO₄ | HNO₃ | Na₂Cr₂O₇ | 20 | 17 | 81 |
| 2 (CD₃CN) | 1 | 2.2 eq. NaIO₄ | HNO₃ | Na₂Cr₂O₇ | 30 | 7 | 68 |
| 2 (CD₃CN) | 1 | 2.2 eq. NaIO₄ | HNO₃ | Na₂Cr₂O₇ | 10 | 17 | 89 |
| 2 (CD₃CN) | 1 | 2.2 eq. NaIO₄ | HNO₃ | Na₂Cr₂O₇ | 0 | 19 | 91 |
| 2 (CD₃CN) | 1 | 2.2 eq. NaIO₄ | HNO₃ | CrO₃ | 20 | 7 | 76 |
| 2 (CD₃CN) | 1 | 2.2 eq. NaIO₄ | H₅IO₃ | Na₂Cr₂O₇ | 20 | 18 | 88 |
| 2 (CD₃CN) | 1 | 2.2 eq. NaIO₄ | HNO₃ | Na₂Cr₂O₇ | 0 | 17 | 90 |
| 0 | 2 | 2.2 eq. NaIO₄ | HNO₃ | Na₂Cr₂O₇ | 20 | 7 | 60 b) |
| 2 (CD₃CN) | 1 | 2.2 eq. NaIO₄ | HNO₃ | Na₂Cr₂O₇ | 0 | 22 | 95 |
| 2 (CD₃CN) | 1 | 3.3 eq. NaIO₄ | HNO₃ | Na₂Cr₂O₇ | 20 | 7 | 80 |
| 2 (CD₃CN) | 1 | 2.75 eq. NaIO₄ | HNO₃ | 2 × Na₂Cr₂O₇ | 20 | 7 + 17 | 98 c) |

eq. = equivalents
a): CH₃CN/H₂O/D₂O = 2.0/0.5/0.5
b): no organic solvent
c): after 7 h, in addition to the 2.2 equivalents of NaIO₄, a further 0.55 equivalent of NaIO₄ was added, and also a further portion of dichromate (in total 4 mol % of Cr).

### EXAMPLE 8

1 mol % of sodium dichromate (2 mol % of Cr) or 2 mol % of CrO₃, and 2.2 equivalents of sodium periodate were introduced into a reaction vessel which was situated in a 20° C. water bath. The mixture was stirred using a magnetic stirrer. Then 1 ml of water (D₂O), 2 ml of d3-acetonitrile and acid, and 1 mmol (112 mg) of 3-heptyn-1-ol were added and the reaction mixture was stirred at 20° C. After the reaction was completed the reaction mixture was filtered off in order to remove insoluble sodium iodate and 3-heptynoic acid was isolated.

The yield of 3-heptynoic acid, depending on the type and amount of acid used, is reported in table 2.

The series of experiments were carried out using an apparatus for carrying out a number of reactions in parallel (Chemspeed).

TABLE 2
Chemspeed experiments: Yield of 3-heptynoic acid formation

| mol % H⁺ | H₂SO₄ | HCl | HNO₃ | pTSA | HBF₄ | CF₃SO₃H | PFTDA |
|---|---|---|---|---|---|---|---|
| 1 | 33 | 26 | 36 | | 34 | | |
| 2 | 45 | 34 | 50 | 49 | 50 | 54 | 65 |
| 5 | 69 | 48 | 75 | 74 | 72 | 82 | 86 |
| 10 | 86 | 68 | 87 | 86 | 91 | 88 | 85 |
| 20 | 81 | 74 | 91 | 90 | 90 | 94 | 94 |

### EXAMPLE 9

In a similar manner to example 7, 1 mol % of sodium dichromate (2 mol % of Cr), or 2 mol % of CrO₃, and 1.1 equivalents or 2.2 equivalents of sodium periodate were introduced into a reaction vessel which was in a 20° C. water bath. The mixture was stirred using a magnetic stirrer. Then 1 ml of water (D₂O), 2 ml of CD₃CN as solvent and acid (corresponding to 20 mol % H⁺), and 1 mmol of alkenol or alkynol were added and the reaction mixture was stirred at a temperature between 0° C. and 30° C. After the reaction was completed the reaction mixture was filtered off to remove insoluble sodium iodate and the corresponding acid or ketone was isolated.

The starting materials used and the reaction parameters (temperature, reaction time and yield) are listed in table 3:

TABLE 3

| Starting | | | 2 mol % | T | t | % |
|---|---|---|---|---|---|---|
| material | Periodate | Acid | of Cr | (° C.) | (h) | yield |
| 4-Heptyn-2-ol | 1.1 eq. NaIO₄ | H₂SO₄ | Na₂Cr₂O₇ | 20 | 20 | 69 a) |
| 3-Butyn-2-ol | 1.1 eq. NaIO₄ | HNO₃ | Na₂Cr₂O₇ | 20 | 19 | 96 |
| 3-Pentyn-1-ol | 2.2 eq. NaIO₄ | HNO₃ | Na₂Cr₂O₇ | 20 | 19 | 88 |
| 3-Butyn-1-ol | 2.2 eq. NaIO₄ | HNO₃ | Na₂Cr₂O₇ | 20 | 16 | 89 |
| 3-Buten-1-ol | 2.2 eq. NaIO₄ | HNO₃ | Na₂Cr₂O₇ | 20 | 16 | 98 |
| 4-Methyl-3- | 2.2 eq. NaIO₄ | HNO₃ | Na₂Cr₂O₇ | 0 | 7 | 86 |
| penten-1-ol | | | | | | |
| 3-Hexyn-2-ol | 1.1 eq. NaIO₄ | HNO₃ | Na₂Cr₂O₇ | 20 | 16 | 95 |

eq. = equivalents
a): 2-phase

1. A method for oxidizing a primary amino alcohol, primary or secondary alkenol or alkynol to the corresponding acid or ketone, wherein a primary amino alcohol, a primary or secondary alkenol or alkynol as a substrate is oxidized to the corresponding ketone in the presence of an equimolar amount or a molar excess, based on the alcoholic hydroxyl groups, of periodate, catalytic amounts of dichromate or CrO₃, and in the presence of an acid, in water, a water/solvent mixture or in a solvent at a temperature of −20° C. to +50° C. to give the corresponding acid or corresponding ketone.

2. The method as claimed in claim 1, wherein the amino alcohol, alkenol or alkynol used are compounds of the formula I 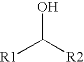
where R1 is either H or a C₁-C₂₀ alkyl radical, an aryl or heteroaryl radical or a heterocycle and R2 is an unbranched or branched, unsubstituted or substituted C₂-C₂₀ alkenyl or alkynyl radical or a C₁-C₂₀ alkyl or aryl radical substituted by one or two amino groups.

3. The method as claimed in claim 2, wherein the amino alcohol used is an aliphatic or aromatic amino alcohol having 2 to 20 carbon atoms, 1 to 2 amino groups and 1 to 2 primary alcoholic hydroxyl groups which may be substituted by acyl, halogen, C₁-C₈ alkoxy, C₃-C₈ cycloalkyl and phenyl.

4. The method as claimed in claim 2, wherein the primary and secondary alkenol and alkynol are compounds of the formula I where R1 is H or an unbranched or branched C₁-C₈ alkyl radical and R2 is C₃-C₁₂ alkenyl or alkynyl radical, where the radicals can be unsubstituted or can be substituted by one or more substituents which are acyl, halogen, C₁-C₈ alkoxy, C₃-C₈ cycloalkyl or phenyl.

5. The method as claimed in claim 1, wherein the 1.5 to 10 molar equivalents of periodate are added, based on the alcoholic hydroxyl groups.

6. The method as claimed in claim 1, wherein the periodate is used in the form of Na, K or Bu₄N salt.

7. The method as claimed in claim 1, wherein dichromate or CrO₃ is used in an amount of 0.1 to 3 mol %, based on the alcohol.

8. The method as claimed in claim 1, wherein the acid is sulfuric acid, HCl, HNO₃, p-toluenesulfonic acid, HBF₄, H₅IO₆, CF₃SO₃H or perfluorotetradecanoic acid or mixtures thereof.

9. The method as claimed in claim 8, wherein, in the oxidation of the amino alcohol, an amount of acid of 1 to 4 molar equivalents, is used and in the case of alkenol and alkynol, an amount corresponding to 1-30 mol % H⁺ of acid is used.

10. The method as claimed in claim 1, wherein the reaction is carried out in water, in a solvent selected from the group consisting of chloroform, dichloromethane, ethyl acetate, diethyl ether, methyl t-butyl ether, dimethoxyethane, 2-methoxyethyl ether, triethylene glycol dimethyl ether, dioxane, THF, acetone, isopropyl acetate and acetonitrile, or in a water/solvent mixture.
